(19) United States
(12) Patent Application Publication
(10) Pub. No.: US 2007/0208390 A1
(43) Pub. Date: Sep. 6, 2007

(54) IMPLANTABLE WIRELESS SOUND SENSOR

(76) Inventors: Jeffrey A. Von Arx, Minneapolis, MN (US); Abhi Chavan, Maple Grove, MN (US); Keith R. Maile, New Brighton, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/276,466

(22) Filed: Mar. 1, 2006

(51) Int. Cl. A61N 1/00

(52) U.S. Cl. 607/32

(57) ABSTRACT

An apparatus and method is presented for an implanted sound sensor wirelessly communicating with an implantable medical device, or with an external monitoring device. The second sensor may be located inside a blood vessel anchored by an expandable stent like device, and may be drug coated. The sound sensor may be a solid-state microphone having a unidirectional characteristic and may be aimed at a selected portion of the heart, lung, or other location. There may be a network of sound sensors forming a local area network with the implantable medical device. The information from the sound sensor may be analyzed, filtered, transformed, compared to a standard and stored in the implantable device, or it may be passed on to an external location. The results of the analysis may be use to initiate a closed-loop treatment by the implantable medical device, such as cardiac pacing or defibrillation.

### TECHNICAL FIELD

This subject matter pertains to implantable medical devices such as cardiac pacemakers, cardioverter/defibrillators and sensors. In particular, the subject matter relates to an apparatus and method for monitoring biological and physiological sounds.

### BACKGROUND

Implantable medical devices (IMD), including cardiac rhythm management devices such as pacemakers and cardioverter/defibrillators, may be capable of communicating data with an external device, such as a programmer, via a radio frequency (RF) or other telemetry link. A clinician may use an external programmer to program the operating parameters of an implanted medical device. For example, the pacing mode and other operating characteristics of a pacemaker may be modified after implantation of the pacemaker into the body in this manner. IMDs may include the capability of bidirectional communication or information may be transmitted to the external programmer from the IMD for monitoring purposes. Data transmitted from an IMD to the external programmer may include various operating parameters and physiological data, either collected in real-time or stored from previous internal monitoring operations. In certain circumstances, the information gathered may warrant an immediate response from in order to avoid potential damage to the monitored body. In such circumstances the IMD may have the ability to make certain changes in the operating characteristics of the IMD within the limits of an allowable range of variables. This may be referred to as a closed circuit control loop, and, for example, may be applied in the case where a dangerous cardiac arrhythmia is detected.

The implanted medical device may have electrical electrodes or leads used to detect or sense the timing and strength of the various portions of a heart beat, or of a long series of heart beats. There may be analysis software included in the implanted device to characterize the measured values from the implanted sensors, to determine if immediate action is required, or whether an immediate alert to the external programmer should be sent, or whether to record the data until a scheduled download occurs.

### SUMMARY

A clinician may use a stethoscope to listen to heart and lung sounds when examining a patient. This may provide valuable information on the conditions found within the body. However, there may be problems with external human monitoring of heart and lung sounds, since the body may be considered as essentially a liquid in which sounds are transmitted easily and efficiently, and the body-to-stethoscope boundary may be a location where the sound waves of interest may be reflected, attenuated and distorted. Thus, listening to body sounds from the outside may result in a failure to capture the desired information due to poor sound transmission at the boundary. Further, it would be beneficial to be able to listen to body sounds, in particular biological and physiologically produced sounds, in a more continuous fashion (i.e., chronically) than is practical with current methods.

Further, listening to body sounds using a human ear from the outside of the body, may result in misinterpreted sound analysis due to human error, the short duration of listening, and the unreliable body to sound detector interface. The present inventors have recognized a need for more continuously, or chronically, detecting biologically generated sounds from inside the body. This permits either electronically storing the sound signatures, or performing an evaluation and comparison on the sounds to determine a current state of the body (e.g., as compared to past values), or to established normal values for the particular body.

This document describes, among other things, an implanted sound sensor, which may be known as a pod, that may be placed in a large blood vessel. The present inventors have recognized a need for an implantable sound detector, for having electronic storage capability within the body, and for sound evaluation capability to enable closed-loop feedback variation within prescribed limits of the operational settings of an implanted medical device, such as a pacer, defibrillator, or other cardiac function management device.

A disclosed implantable medical device (IMD) has an acoustic wave detector that is communicatively coupled to an implantable medical device housing. The acoustic wave detector may be situated to detect biologically generated sounds from areas of interest. The IMD may include an electronic circuit configured to communicate with either an external programmer, an external communications center, or an external emergency center, such as by using RF, inductive, or other telemetry. The IMD may include a cardiac function management device, a diagnostic analysis circuit, a memory circuit, or a closed-loop controller for adjusting the settings and performance of the implantable device.

The acoustic wave detector may be formed of a biocompatible material and implanted in the body, such as adjacent to, or even remote from the IMD. In various examples, the acoustic wave detector is coupled to the IMD housing by modulated electromagnetic radiation, modulated radio frequency radiation, modulated acoustic radiation, modulated ultrasonic waves, modulated e-field, electrical wires or by inductive coupling. There maybe a plurality of such acoustic wave detectors located either in or on the body, each of the detectors being a part of a communications network that includes the IMD as either a communications hub, or as a controller, or an analysis and storage center, or as a port for communications to an external location, such as a physician. The acoustic wave detector is typically battery-powered, and may be recharged by externally applied ultrasonic waves acting on a ultrasonic wave detector/generator, or by inductive coupling through the skin.

The acoustic wave detector is typically made of a biocompatible material or located inside a hermetic biocompatible pod. In certain examples, the pod may be formed of titanium or glass. In one illustrative example, the pod is formed of titanium having a wall thickness of between 0.001 to 0.002 inches—for example, thin enough such that a desired acoustic wave will pass easily through the walls. In an alternative example, the pod is formed of titanium having a greater wall thickness, such as between 0.011 to 0.012 inches, and having a diaphragm portion near the acoustic wave detector, the diaphragm portion having a thickness of between 0.001 to 0.002 inches so as to allow acoustic waves to pass through the diaphragm to reach the detector (or the diaphragm may form part of the detector). The pod may have a shape such as a cylinder, a can, a pill, a capsule or a tube, and may be small enough to fit within a blood vessel, such as the pulmonary artery. For example, the pod may have a diameter or similar dimension of less than 3.5 mm in at least one direction so as to reduce or minimize interference with blood flow. In another example, the pod may have a diameter or similar dimension of less that 2.0 mm in at least one direction, such as for implantation in a blood vessel smaller than the pulmonary artery.

In certain examples, the pod is anchored to the desired location inside the blood vessel by an expandable stent or stent-like mesh. The stent may be manually expanded by a catheter balloon or it may be self expanding. In certain examples, the stent is drug-coated. Examples of drugs used in stent coatings include Sirolimus, Paclitaxel, and Everlimus. The pod is typically attached to the mesh, such as in-between an inside wall of the blood vessel and an outside wall of the mesh, or inside the inner wall of the mesh, for example. In various examples, the acoustic detector pod may be introduced into a blood vessel, an esophagus, a trachea, a bronchus, a lung, a peritoneum, a pericardium or one of the alimentary organs.

In certain examples, the pod encloses the acoustic wave detector, an ultrasonic communication device, an acoustic impedance matching material (e.g., to improve the conduction efficiency of the detector), and a rechargeable battery. The acoustic impedance matching material may be a silicone gel or oil, or one or more other types of oils, such as hydrocarbon or fluorocarbon liquids, that substantially completely fills the pod, or at least an area about the enclosed acoustic wave detector. The acoustic impedance matching material typically has a viscosity that is selected to match a desired acoustic wavelength, such as to provide high acoustic conductance across the body-to-microphone interface, preferably with losses of no more than 80%. In one illustrative example, the acoustic wave detector is directional, such that it can be aimed at a selected location, such as the heart or a lung. The directional sound detector may be aimed in any direction during implantation, for example by turning the delivery catheter. Fluoroscopic markers may be used to allow a physician to see the direction of the sensor during implantation. Sound level measurements during implantation may also be used to help determine the proper detector placement. In another example, the acoustic detector is substantially omni-directional. The acoustic wave detector is typically used to monitor one or more of a heart sound, a mitral valve regurgitation sound, an S3 heart sound, a lung sound (e.g., rasps, rales, cough, etc.) or blood vessel flow sounds (e.g., resulting from an aneurism or vessel constriction).

The implantable medical housing typically receives biological and physiological sound information transmitted from the pod. It may store or process such sound information. For example, the IMD may store the heart or other recorded physiological sounds, transmit information about such sounds to an external programmer, perform a fast Fourier transform on such sounds, compare current sounds to previously-recorded sounds in the same patient, compare current sounds to a stored template sound, perform an analysis of detected sounds, or perform a closed-loop regulation of an interventional device, such as a cardiac function management device, which may be included in the IMD.

The biological sound detector may be remotely communicatively coupled to the IMD in a body, such as by using modulated ultrasonic waves. The IMD in the body may also include an accelerometer, a position detector, a temperature detector, or logic, such as to evaluate the detected biological sounds in conjunction with the measured body position. This may be useful for a patient with breathing difficulty in a supine position, or with sleep apnea. The accelerometer can be used to determine the body's physical posture, such as standing, sitting, prone, supine, or recumbent. The IMD will typically include a processor with analysis software that may use the posture information together with the sound information, such as to attenuate the influence of changes in posture on the sound information, for example. The IMD may then evaluate posture-corrected sound data, for example, and may communicate with an external programmer, an external communications center, or an external emergency center, such as by using RF or another communication technique.

In various examples, the biological sound detector will include a solid-state microphone, a unidirectional microphone, a multidirectional microphone, an omni-directional microphone, a carbon microphone, a piezoelectric microphone, a piezoresistive microphone, or a capacitive microphone. The sound detector is typically in a sealed pod with an ultrasonic or other transmitter or transceiver and a battery. The biological sound detector (pod) may be surrounded with a fluid, gel, or other acoustic impedance-matching material to improve acoustic coupling with the body.

Other aspects of the present systems and methods will be apparent upon reading the following detailed description and viewing the drawings that form a part thereof

### BRIEF DESCRIPTION OF THE DRAWINGS

FIG. 1 illustrates an example of a group of sound monitors in a body;

FIG. 2 illustrates an example of a cross sectional view of a sound detector implanted in a blood vessel; and

FIG. 3 illustrates an example of cross sectional view of an implantable medical device.

### DETAILED DESCRIPTION

In the following detailed description, reference is made to the accompanying drawings that form a part hereof, and in which is shown, by way of illustration, specific embodiments in which the present subject matter may be practiced. These embodiments are described in sufficient detail to enable those skilled in the art to practice the subject matter, and it is to be understood that the embodiments may be combined, or that other embodiments may be utilized and that structural, mechanical, logical and electrical changes may be made without departing from the scope of the present subject matter. The following detailed description is, therefore, not to be taken in a limiting sense, and the scope of the present subject matter is defined by the appended claims and their equivalents.

The present inventors have recognized, among other things, a need for an implantable biological and physiological sound sensor, for example, which can be separately located from an implantable medical device (IMD), such as a cardiac function management device (which may be known as a pulse generator, PG), and which can have wireless communication with such an implantable medical device. In certain examples, this will enable a closed loop response to physiological sound information. In certain examples, this will permit reporting of physiological sound information to an external location, such as for clinician review.

The sound sensor may be located within the IMD, outside the IMD, or preferably, at a location remote from the IMD. The location will typically depend upon what physiological sounds the biological sound sensor is intended to listen to. For example, it may be useful to listen to heart sounds using a microphone located in the pulmonary artery. Similarly, it may be useful to listen to lung sounds using an axillary microphone location. If the biological sound sensor is located inside a major blood vessel, it may be anchored in place by an expandable stent-like device, such as a self-expanding stent, or a catheter balloon expanded stent. The stent may be drug coated, such as to inhibit or prevent local stenosis, for example using Everlimus (Guidant's Xience V stent), Paclitaxel (Boston Scientific Taxus stent), and Sirolimus (J&J Cypher stent), or other drugs.

The biological sound sensor may be a solid-state microphone. In certain examples, the microphone has a directional sound-receiving characteristic. By aiming a directional microphone at a selected portion of the heart, lung, or other location generating sounds of interest, physiological sound noise from other locations may be reduced. The biological sound sensor may be aimed during implantation using the delivery catheter to place the sensor, and adjusting the direction in response to received sound levels, or radiographic markers may be used to allow visualization of the sensor. There may be more than one sound sensor implanted in a body, which allows each biological sound sensor to listen to a different area of interest, if desired. Multiple biological sound sensors can form a local area network with an implantable cardiac function management device or other implantable medical device. This would permit the various biological sound sensors to communicate with the implantable medical device as desired. The sound information may be analyzed, filtered, transformed, compared to a template or recent sound, stored in volatile or non-volatile memory, or communicated to an external location. The implantable medical device will typically perform such processing, storage, or communication to an external location. The implantable medical device may also use the biological sound information to initiate or adjust an electrostimulation or other therapy by the implantable medical device, such as by implementing a closed-loop control system.

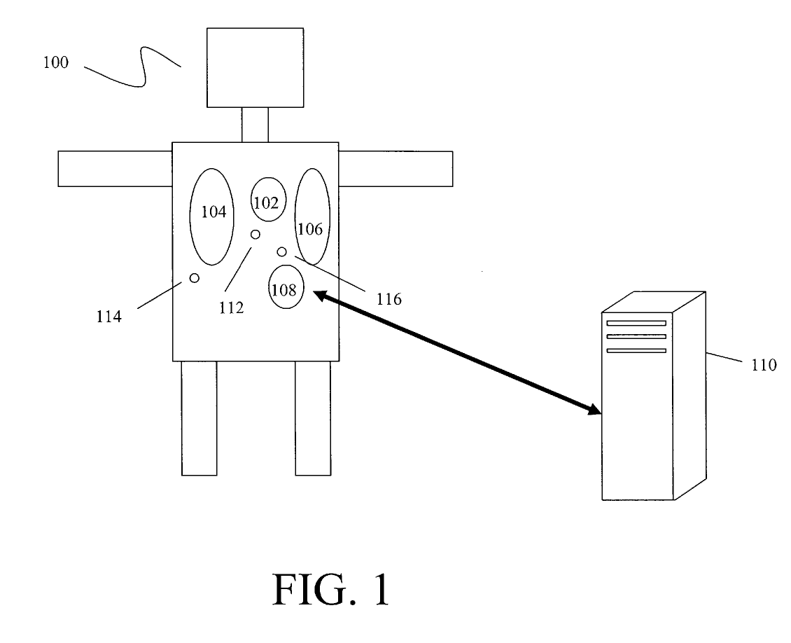

FIG. 1 illustrates an example of a cutaway view of a group of three sound sensors in a human body. The body 100 includes a number of locations of potential interest for sound monitoring. For example, the heart sounds produced by the heart 102 may include such information as heart rate, spontaneous ventricular contraction, S1, S2, S3, or S4 sounds, mitral valve regurgitation, fibrillation, or contractility. Such information may be used to diagnose a patient, or to initiate or modify medical treatment for the patient. The right lung 104 and the left lung 106 may also produce clinically-useful sounds, such as coughs, rasps, rales, or wheezes. Information obtained from one or more physiological sounds may be transmitted to an implantable medical device 108, which may be a cardiac function management device, such as a pacer, a defibrillator, or a cardiac resynchronization therapy device, and which may be separately located from the biological sound sensors. In certain examples, the implantable medical device 108 includes electronic telemetry circuitry, such as a transceiver, which may include a transmitter, a receiver, or both a transmitter and a receiver. This permits radio frequency or other communication to an external device, such as local external programmer 110. The external device, in turn, may communicate with a remote patient monitoring system, such as by RF, conventional telephony, or a communications network.

Each of the locations of interest, the heart 102, the lungs 104 and 106 in this illustrative example, may use a separate biological sound detector. In this illustrative example, sound detector 112 is positioned at a location favorable for obtaining readings for all or a selected portion of the heart 102. Similarly, sound detectors 114 and 116 are positioned to obtain favorable biological sound readings from the right and left lung respectively. The sound detectors 112, 114 and 116 may be either omnidirectional microphones, or unidirectional microphones oriented for receiving sound information from a region of specific interest. For example, the acoustic information may be transmitted to the IMD 108 as part of a local area network, or another method of group communication. In certain examples, the biological sound detectors 112, 114 and 116 communicate wirelessly with the IMD 108 using bidirectional communication. Such wireless communication may use communication means such as modulated ultrasonic waves, or modulated electromagnetic waves such as radio frequency modulation.

The IMD 108 may analyze the biological sound information by various techniques, such as by using Fast Fourier Transform (FFT), other mathematical transformations, filtering, comparison with one or more template or actual time or frequency domain acoustic patterns. The IMD 108 may include electronic circuitry to declare that a particular physiological condition has occurred, to determine the correct response (e.g., cardiac defibrillation in response to a detected fibrillation), or to transmit an alert or other information to an external location, such as by using of radio wave or other communication. The external location may include a processor to perform similar analysis or processing of the sound information as the IMD 108. If the IMD 108 does not detect an immediate problem, it may store the acoustic information, analyzed or in original form or both, in an included electronic storage medium, such as RAM, flash memory, or in a magnetic storage medium.

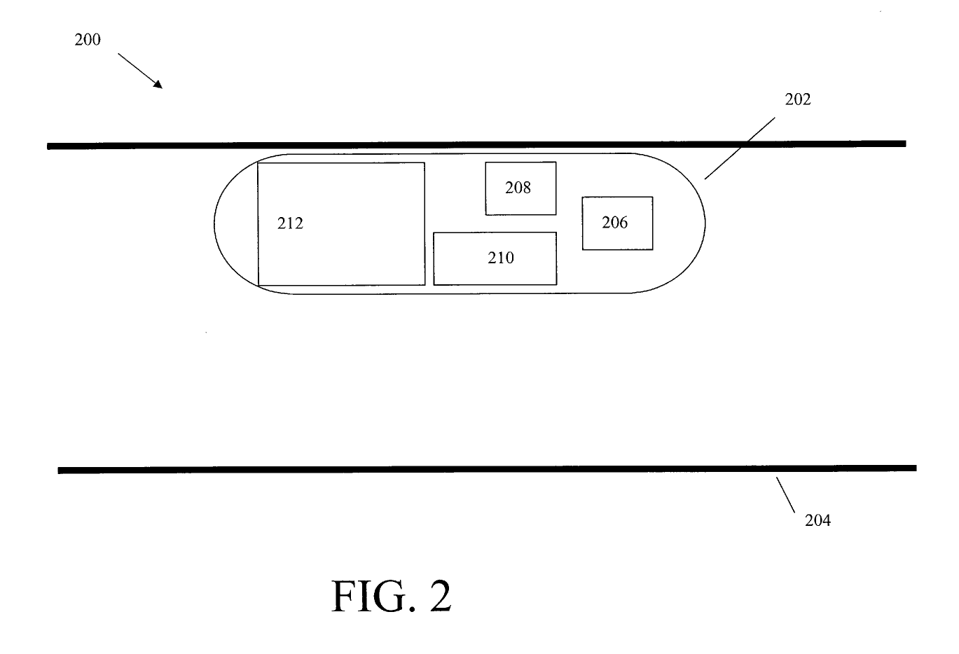

FIG. 2 illustrates a cross sectional view of one of the sound detectors, such as 112, 114 or 116, of FIG. 1, implanted in a major blood vessel. In this illustrative example, the sound detector is shown in the form of a cylindrical pod having rounded ends, but other shapes are also possible. The specific form of the sound detector pod may be modified to fit in any desired location, such as near a pectoral or other muscle, in an internal organ such as a liver, or inside a blood vessel, as shown in this example.

An illustrative biological sound detector system 200 is shown with a sealed sound detector pod 202 attached to an inside surface of a blood vessel 204. The pod 202 may be attached to the vessel using a stent (or like-structure), which typically includes an open-ended wire tube that may be expanded upon insertion at the desired position, such as by using a balloon catheter. Alternatively, a self-expanding stent may be used, or other methods known in the art. The pod 202 may be attached to the stent either before or after the stent is inserted and anchored in position by means of adhesives, clamps and mechanical attachments. To prevent or inhibit the tissue of the inside wall of the blood vessel 204 from reacting to the presence of the foreign body represented by the pod 202, the stent may be drug coated using any of a number of well known materials.

The pod 202 typically includes a pod shell formed of a bio-compatible material such as glass or titanium, and will typically have at least a portion of the pod shell that is conductive to acoustic waves. One or more portions of the inside of pod 202 may be filled with a liquid or gel that is conductive to acoustic waves. The acoustic impedance (affected by the viscosity, density and other physical characteristics) of the liquid or gel may be selected to obtain good or maximum acoustic conduction, for example, greater than 80% efficient acoustic transmission across the blood-pod interface. Illustrative examples of liquid and gel materials include silicone, hydrocarbon or fluorocarbon materials.

The walls of the pod 202 are generally thick enough to maintain the desired shape, in this illustrative example, a streamlined shape that provides low or minimum interference with blood flow. At least a portion of the walls of the pod 202 is not so thick that acoustic conduction efficiency declines beyond acceptable limits. In an illustrative example, the pod 202 includes a titanium wall that is about 0.012 inches thick, with a thinner portion of the wall being about 0.002 thick. The thinner portion forms an acoustically transmissive membrane near an acoustic pickup or microphone within the pod. The actual thickness of the pod 202 walls needed to maintain the desired shape will depend upon the forces the pod 202 needs to withstand, and whether the interior portion of the pod (about any sound detector, battery or other internal components carried by the pod) is essentially completely filled with an incompressible material such as fluorocarbon oil.

Non-biocompatible materials may be used inside the sealed pod 202. Non-biocompatible materials may even be used for the pod walls if suitably sealed with a bio-compatible material, such as a layer of polytetrafluoroethylene (PTFE), expanded polytetrafluoroethylene (ETFE), polyetheretherketone (PEEK), parylene, silicone, polyurethane, Tecothane, aromatic polyether thermoplastic, or glass. In this illustrative example, the pod 202 could be formed using a thinner and more acoustically conductive material, which is also uniform, well-controlled, and more inexpensive than biocompatible titanium. The pod 202 includes an acoustic pickup or microphone 206. The microphone 206 may be any type of acoustic pickup, such as an electronic microphone, a piezoelectric acoustic pickup, piezoresistive acoustic pickup, a carbon microphone, a capacitor or other microphone system, and may be either omnidirectional or directional. The microphone is coupled to a transmitter or transceiver for unidirectional or bidirectional communication with the implantable medical device 108 of FIG. 1. In certain examples, the microphone and ultrasonic transmitter or transceiver are both included in a single pod. The transmitter or transceiver 208 is typically controlled by circuitry 210 and powered by battery 212.

In certain illustrative examples, the battery 212 is rechargeable, such as by occasional inductive coupling to receive power from an external source, or by externally applied ultrasonic waves that are picked up by the ultrasonic element 208, converted into an electrical charge, and directed to the battery 212. The circuitry 210 may also include a controller or communications interface with a wireless local area network, such as found in patent application 10/913,118 filed on Aug. 4, 2004, entitled System and Method for Providing Digital Data Communications Over a Wireless Intra-Body Network, incorporated herein, where multiple sensor pods communicate with an implantable medical device (IMD), to coordinate communication by the various sensor pods. This will avoid collisions of data from the various sensor pods.

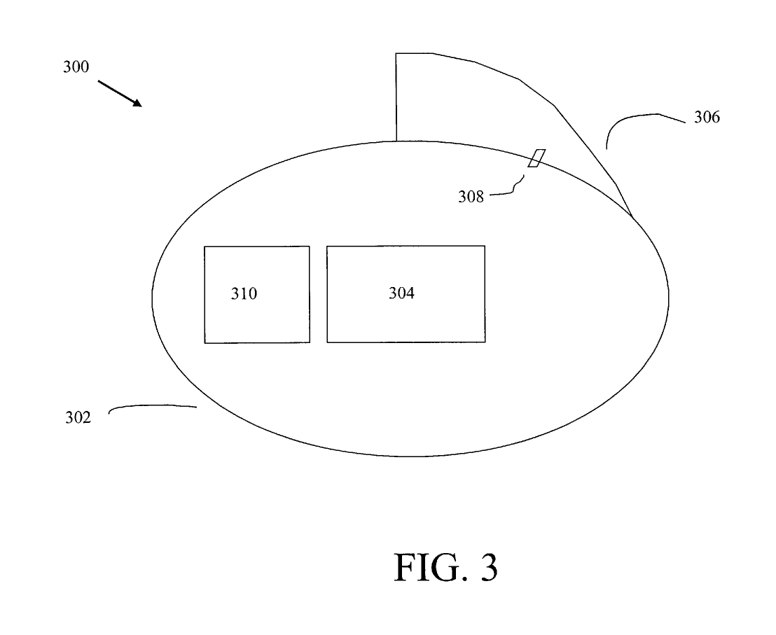

FIG. 3 is a illustrative view of an implantable medical device 300, having a hermetically sealed housing 302, which may include a conductive surface over all or part of its surface. The IMD housing 302 typically includes electronic circuitry 304 for providing particular functionality to the device such as cardiac function management, physiological monitoring, drug delivery, or neuromuscular stimulation as well as RF telemetry or other communication circuitry. The device 300 typically includes a header 306 attached to the IMD housing 302, such as to receive one or more intravascular leads or the like. The header 306 may include one or more electrical feedthroughs to conduct signals between the electrodes on the leads and circuitry within the housing 302. A feedthrough 308 will typically provide an electrically isolated conductive path through a wall of housing 302, while preserving the hermetic seal for the environment within an interior of the housing 302. The portion of the IMD housing 302 containing the feedthroughs typically includes certain regions of insulating material to avoid shorting of the feedthroughs to the IMD housing or to each other. There will typically be one feedthrough 308 for each electrical signal that leaves the IMD housing 302, such as for connecting to an electrode in association with a portion of a patient's heart for sensing an intrinsic cardiac signal or for delivering an electrical stimulation pulse or shock or for connecting the circuitry 304 to an antenna located outside of the IMD housing 302, such as at a location that is either inside or outside of the header 306.

The IMD housing 302 may also include an ultrasonic receiver or transceiver 310 for either unidirectional or bidirectional communication with one or more of the biological sound sensor pods 200 of FIG. 2, implanted in the body. In certain examples, the transceiver 310 is located outside of the housing 302 with one or more electrical signals transferred to the communication circuitry 304 through one or more wired connections 308 in the IMD header 306. The circuitry 304 controls communication with one or more of the single sound detector pod, a network of such pods, an external programmer console or other local external device, or a remote patient management server.

In certain examples, the internal communication between the IMD housing 302 and the separate sound detector pod(s) typically use one or more modulated ultrasonic signals, such as disclosed in patent application 10/888,956 filed Jul. 9, 2004, entitled System and Method of Acoustic Communication for Implantable Medical Device, incorporated herein. The communication between the IMD housing 302 and an external location is typically performed using modulated radio frequency or other electromagnetic or magnetic signals. The circuitry 304 may also include logic and memory elements, such as for comparing one or more of the biological sound recordings to one or more previously stored recordings of the specific individual's normal or abnormal heart or lung sounds, or for comparing a current sound to a template or other standardized biological sound pattern. In certain examples, a deviation from an allowable specified limit will trigger an alert to an external location, or a responsive therapy delivery or adjustment by the implantable medical device. Such responsive therapy may include cardiac function management therapy, such as bradycardia pacing, cardiac resynchronization, cardioversion or defibrillation, anti-tachyarrhythmia pacing, or the like. The circuitry may also include logic or performed instructions that process the received sound information, such as an FFT, high pass, low pass or band pass filtering, or frequency analysis, such as to determine if a physiological condition is detected. If the detected physiological condition is not so severe as to warrant providing an immediate alert to an external location, the circuitry 304 may store in an accompanying memory element either or both of the recorded sound and information obtained from the analysis. Such information can then be transmitted to an external location, if desired, at a later time, such during prescribed time periods.

The housing 302 may also contain one or more accelerometers or position sensors, such as to determine the recent activity level of the patient and the position of the body, including, for example, one or more of supine, prone, recumbent, sitting, standing, or leaning, or to provide information on position-dependent conditions, such as night coughing or wheezing, exercise-induced dyspnea or heart rhythm abnormalities.

The above description is intended to be illustrative, and not restrictive. Many other embodiments will be apparent to those of skill in the art upon reviewing the above description. The scope of the present invention should not be limited to the described embodiments, and is set forth in the following claims.

What is claimed is:

1. A system, comprising:
an implantable housing including a memory circuit and an electronic communication circuit configured to communicate from within a body to an external location; and
a separate implantable biological or physiological sound detector communicatively coupled to the implantable housing when the sound detector and the implantable housing are both implanted in a body.

2. The system of claim 1, wherein the implantable housing comprises at least one of a cardiac pacer, a cardiac defibrillator, a cardiac resynchronization device, a diagnostic analysis circuit, a memory circuit and a closed-loop controller.

3. The system of claim 1, wherein the sound detector includes a transceiver to communicate with the implantable housing using at least one of a modulated electromagnetic signal, a modulated radio frequency signal, a modulated acoustic signal, a modulated ultrasonic signal, a modulated e-field, an electrical wire, and an inductive coupling signal.

4. The system of claim 3, comprising a plurality of the sound detectors in communication with the implantable housing when the plurality of the sound detectors and the implantable housing are implanted within the body.

5. The system of claim 1, wherein the sound detector comprises a battery that is rechargeable by at least one of externally applied ultrasound and inductively-coupled power transmission.

6. The system of claim 1, comprising a biocompatible pod enclosing the sound detector.

7. The system of claim 6, wherein at least one of:
the wall includes a wall thickness of between 0.001 to 0.002 inches; and
the wall includes a wall thickness of between 0.011 to 0.012 inches and the wall further includes a diaphragm portion disposed near the sound detector having a thickness of between 0.001 to 0.002 inches.

8. The system of claim 6, wherein the pod has a shape that is selected from a cylinder, a can, a pill, a rectangular solid, a capsule and a tube.

9. The system of claim 8, wherein the pod has a major dimension and a minor dimension, the minor dimension being less than 2.0 mm.

10. The system of claim 9, wherein the pod is sized and shaped to permit the pod to be disposed inside a blood vessel.

11. The system of claim 6, wherein the pod includes an anchor, attached to the pod, to anchor the pod to a selected position inside the body.

12. The system of claim 11, wherein the anchor comprises one of an expandable stent-like mesh, and a self-expanding stent-like mesh.

13. The system of claim 10, wherein the anchor comprises a drug-coated stent-like mesh.

14. The system of claim 6, wherein the pod is sized and shaped to permit the pod to be disposed at a location selected from inside an esophagus, trachea, bronchus, lung, peritoneum, pericardium and an alimentary organ.

15. The system of claim 6, wherein the pod includes the sound detector, an ultrasonic communication device for communicating with the implantable housing, an acoustic impedance matching material, and a rechargeable battery.

16. The system of claim 15, wherein the acoustic impedance matching material is selected from a list including a silicone gel, a silicone liquid, a hydrocarbon fluid, a fluorocarbon fluid, and mixtures thereof, wherein the impedance matching material substantially completely fills a portion of the pod near the sound detector and has a viscosity selected to match a desired acoustic wavelength.

17. The system of claim 15, wherein the sound detector includes a directional acoustic wave detector to be aimed at a selected portion of the body, and the sound detector is configured to monitor at least one of heart sounds, mitral valve regurgitation, S3 heart sounds, lung sounds, rasps, rales, cough, and blood vessel sounds.

18. The system of claim 1, wherein the implantable housing comprises a signal processing circuit configured to perform at least one of: a fast Fourier transform, a comparison of a later heart sound to an earlier heart sound, a comparison of a detected heart sound to a stored template heart sound.

19. The system of claim 1, wherein the sound detector comprises a microphone.

20. The system of claim 1, wherein the implantable housing comprises at least one of an accelerometer, a position detector, a temperature detector, and a closed-loop regulator to regulate a therapy at least partially in response to information in detected waves.

21. A system comprising:
an implantable housing including:
a cardiac function management circuit; and
an electronic communication circuit configured to perform two way communication via a modulated radio frequency carrier with an external device; and
a first ultrasonic communication circuit; and
an implantable biological sound detector, adapted to be located remote from the implantable housing, the sound detector comprising a second ultrasonic communication circuit for ultrasonic communication with the first ultrasonic communication circuit, the remote sound detector including an anchor to secure the remote sound detector at a desired location.

22. The system of claim 21, wherein the sound detector comprises a biocompatible covering having a diameter of less than 2 mm, the covering enclosing the sound detector, an ultrasonic transceiver, and a battery.

23. The system of claim 22, wherein the biocompatible covering comprises at least one of a titanium shell, a glass shell, and a plastic film, and wherein the biocompatible external covering includes at least one location having a high acoustic conductance.

24. The system of claim 23, wherein the sound detector includes a unidirectional microphone aimed at the location having a high acoustic conductance.

25. A method comprising:
detecting internal physiological acoustic waves using a first implantable medical device at a first location within a body; and
wirelessly communicating information obtained from the acoustic waves from the first implantable medical device at the first location to a separate second implantable medical device at a second location within the body.

26. The method of claim 25, comprising using the information for at least one of closed-loop delivering of therapy from the second implantable medical device and communicating the information to an external device.

27. The method of claim 25, comprising disposing the first implantable medical device in a blood vessel of the body, and disposing the second implantable medical device in a subcutaneous pectoral region of the body.

28. The method of claim 25, comprising disposing a directional microphone aimed at a selected one of a heart, a lung and a blood vessel.
